(19) United States
(12) Patent Application Publication
(10) Pub. No.: US 2025/0062035 A1
(43) Pub. Date: Feb. 20, 2025

(54) SYSTEMS AND METHODS FOR IDENTIFYING CLINICAL CONDITIONS FROM HEALTH RECORDS

(71) Applicant: UST GLOBAL (SINGAPORE) PTE. LTD., Singapore (SG)

(72) Inventors: Adnan Masood, Temple Terrace, FL (US); Geethi Nair, Aliso Viejo, CA (US); Alla Abdella, Aliso Viejo, CA (US)

(21) Appl. No.: 18/449,401

(22) Filed: Aug. 14, 2023

(51) Int. Cl. G16H 50/70; G16H 10/60; G16H 50/20

(52) U.S. Cl. CPC G16H 50/70; G16H 10/60; G16H 50/20

(57) ABSTRACT

A system is configured to: (a) analyze data records using word embeddings to produce vectors indicative of similarity scores for words or phrases in the data records relative to electronically stored reference data; (b) compute shapeley additive explanation (SHAP) values to analyze and rank contributions of the words or phrases to the similarity scores from the word embeddings; (c) use a language model trained on a vocabulary used in the data records to identify contextual insights that are not explicitly disclosed in the data to improve accuracy of the word embeddings; and (d) iteratively and dynamically adjust or update the model using human-in-the-loop based reinforcement learning from human feedback by: receiving an input representative of a modification to at least one of the contributions determined by the SHAP computation, and responsive to receiving the input, adjusting at least one of the ranks of the word embeddings in the model.

### TECHNICAL FIELD

The present disclosure relates to document understanding systems, and more specifically, to systems and methods for identifying clinical conditions or diagnoses from longitudinal patient records and reference data.

### BACKGROUND

Machine learning techniques are used in various applications for predicting future states of a system or for classifying information. In some instances, machine learning can help people make better decisions by incorporating factors that a human mind may not have considered. For example, humans may take into account a few salient factors when making a decision while a computer can take into account hundreds, if not thousands, of factors. Machine learning has been shown effective in image and speech recognition, automated trading strategies for financial instruments, learning associations to develop insights in consumer buying behavior, classifying consumers or objects into different categories, predicting a loan applicant's default risk, etc.

Machine learning techniques are increasingly being applied to many more applications, thus increasing its impact on society. The healthcare space is a heavily-regulated space, where protocols are developed with specific regulations in mind, and in some cases, tools used by healthcare providers have to be approved by regulators. For example, privacy requirements, enforced through regulation, can restrict use and sharing of patient data. In another example, patient protection measures can require certain information be communicated to patients by licensed physicians, thus limiting information that can be shared with patients by unlicensed workers. The regulatory framework in healthcare can limit algorithms that can be used when analyzing health data and health records, and furthermore, can limit communicating the results of those analyses to patients. The present disclosure provides systems and methods for building healthcare systems that are compatible with regulatory frameworks while leveraging machine learning techniques and algorithms in a manner that allows a level of probity and explainability of results obtained by the algorithms.

### SUMMARY

According to some implementations of the present disclosure, a system for conducting feature importance analysis is provided. The system includes a processor and a non-transitory computer readable medium storing instructions such that when the instructions are executed by the processor, the system is configured to: (a) provide a deep learning model; (b) analyze data records using word embeddings to produce vectors indicative of similarity scores for words or phrases in the data records relative to electronically stored reference data; (c) compute shapeley additive explanation (SHAP) values to analyze and rank contributions of the words or phrases to the similarity scores from the word embeddings; (d) use a language model trained on a vocabulary used in the data records to identify contextual insights that are not explicitly disclosed in the data to improve accuracy of the word embeddings; and (e) iteratively and dynamically adjust or update the model using human-in-the-loop based reinforcement learning from human feedback (RLHF) by: (i) receiving from a human via an electronic human-machine interface (HMI) an input representative of a modification to at least one of the contributions determined by the SHAP computation; and (ii) responsive to receiving the input, adjusting at least one of the ranks of the word embeddings in the model.

According to some implementations of the present disclosure, a method for conducting feature importance analysis is provided. The method includes: (a) providing a deep learning model; (b) analyzing data records using word embeddings to produce vectors indicative of similarity scores for words or phrases in the data records relative to electronically stored reference data; (c) computing shapeley additive explanation (SHAP) values to analyze and rank contributions of the words or phrases to the similarity scores from the word embeddings; (d) using a language model trained on a vocabulary used in the data records to identify contextual insights that are not explicitly disclosed in the data to improve accuracy of the word embeddings; and (e) iteratively and dynamically adjusting or updating the model using human-in-the-loop based reinforcement learning from human feedback (RLHF) by: (i) receiving from a human via an electronic human-machine interface (HMI) an input representative of a modification to at least one of the contributions determined by the SHAP computation; and (ii) responsive to receiving the input, adjusting at least one of the ranks of the word embeddings in the model.

The foregoing and additional aspects and implementations of the present disclosure will be apparent to those of ordinary skill in the art in view of the detailed description of various embodiments and/or implementations, which is made with reference to the drawings, a brief description of which is provided next.

### BRIEF DESCRIPTION OF THE DRAWINGS

The foregoing and other advantages of the present disclosure will become apparent upon reading the following detailed description and upon reference to the drawings.

FIG. 1 illustrates a block diagram of a system for identifying clinical conditions, according to some implementations of the present disclosure.

FIG. 2 illustrates a process for training a machine learning model, according to some implementations of the present disclosure.

FIG. 3 provides an example longitudinal health record (or longitudinal patient record), according to some implementations of the present disclosure.

While the present disclosure is susceptible to various modifications and alternative forms, specific implementations have been shown by way of example in the drawings and will be described in detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

### DETAILED DESCRIPTION

Machine learning techniques, being widely used in various applications, affect human lives and livelihood. For example, machine learning is used to determine trading strategies for financial instruments, thus having an effect on individuals' retirement funds and financial markets generally. Machine learning techniques are used to provide recommendations on whether a convicted felon up for parole is likely to re-offend. Machine learning techniques are used for internet search results, thus exposing consumers to specific information based on keywords provided by the consumers. Different consumers using the same keywords may end up with different search results, due to several other factors taken into account by the machine learning algorithm. Due to the overall impact machine learning algorithms have on society, it is beneficial to determine whether some factors that machine learning takes into account when providing a recommendation is wise. For example, knowing that the machine learning algorithm considers the most salient symptoms when performing a medical diagnosis can better inform individuals of drawbacks associated with the algorithm and possible ways of improving the algorithm. Explainability in machine learning systems is crucial especially when these systems are used in a heavily regulated space like healthcare industry.

The average life expectancy across OECD countries is around 81 years. The collective population of OECD countries is over 1 billion individuals. OECD countries are used as an example for illustration purposes, but other divisions or groupings can be used to make a similar point. Each individual, in the over 1 billion, has a health history with different information populated within that history. As the individual lives and visits health facilities, health data associated with the individual continues to grow. The health data does not wait for regulations to catch up on how to deal with such data explosion. As health data is accumulated over time across the population, inability to properly analyze and assess implications of the accumulated health data can result in poorer health outcomes for patients over time. Applying machine learning algorithms to this data explosion may be necessary to extract insights that a quick doctor's appointment may not provide. Depending on the patient's age, doctors are not equipped to digest the entirety of patient's medical history to determine the best diagnoses.

For example, a patient may have a longitudinal health record that includes medical history of the patient from birth. Longitudinal health records are difficult for doctors (or healthcare specialists) to analyze in detail because the longitudinal health records are detailed and can contain unstructured data, including blocks of text and notes. Healthcare specialists typically have a limited duration to spend with patients, and relying on a human to analyze each patient's longitudinal health record before making recommendations is unfeasible. Healthcare specialists typically zero in on a few items and may miss important information that could be indispensable to properly diagnosing or treating a patient. Misdiagnosing or providing unnecessary treatments can be expensive endeavors and can complicate the patient care workflow. For example, misdiagnosing can result in providing a wrong diagnosis (e.g., a diagnostic code, a wrong treatment code, an ICD-10 code, etc.). A wrong ICD-10 code can result in payment delays if the patient's insurance company does not cover the ailment disclosed in the wrong ICD-10 code. Embodiments of the present disclosure provide a solution for properly analyzing longitudinal health records in the healthcare space.

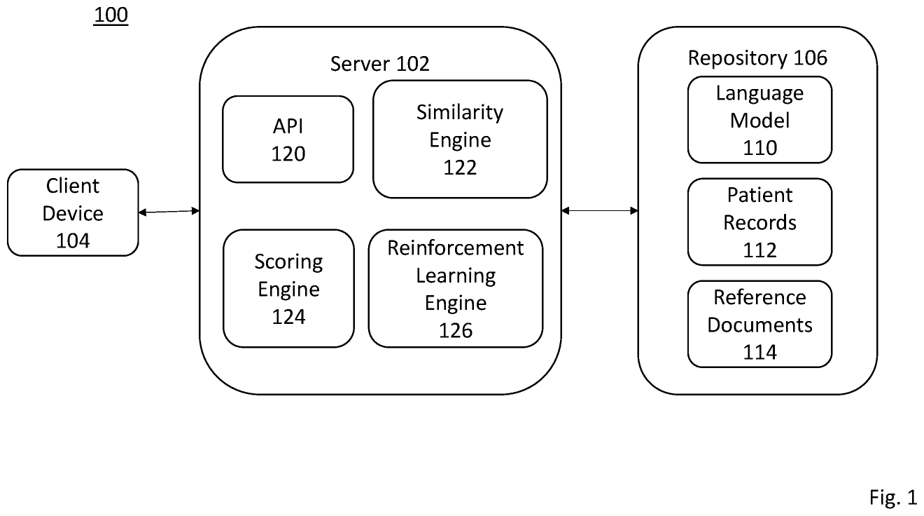

FIG. 1 illustrates a block diagram of a system 100 for identifying clinical conditions, according to some implementations of the present disclosure. The system 100 includes a server 102, a client device 104, and one or more repositories 106 for storing information. The server 102 and the client device 104 are computing devices with at least one processor, memory, storage device, and network interface. Examples of the client device 104 include a laptop computer, a desktop computer, a smart phone, a tablet, a phablet, a personal digital assistant (PDA), a smart television, a smart speaker, etc. The server 102 can include one or more computing devices to perform functions described in the present disclosure.

The one or more repositories 106 can store a language learning model or language model 110, data records or patient records 112, and reference data or reference documents 114. The one or more repositories 106 can store intermediate calculations and other data used by the server 102. The one or more repositories 106 can be housed at a separate location from the server 102 and/or owned by a different entity than the server 102.

The server 102 is configured to receive data records or portions of the patient records 112 from the client device 104. The server 102 can connect to an electronic health record to receive the data records. The server 102 can store the received portions of the patient records 112 in the repository 106 to update patient information (i.e., the patient records 112) included in the repository 106. The server 102 can provide various clinical conditions to the client device 104, based on the received data records associated with the patient. In some implementations, a user of the client device 104 can provide feedback to the server 102 based on the clinical conditions provided by the server 102. For example, the user of the client device 104 is a doctor that can provide feedback on whether the clinical condition identified by the server 102 is plausible, given the doctor's experience. Because the user of the client device 104 can provide feedback to the server 102 to help guide the server 102, knowing which features or words that the server 102 used in determining the clinical condition is important. In some implementations, the feedback provided by the doctor is used to demote or promote words used in determining the clinical conditions.

The server 102 includes an application programming interface (API) 120, a similarity engine 122, a scoring engine 124, a reinforcement learning engine 126. Each of the API 120, the similarity engine 122, the scoring engine 124, and the reinforcement learning engine 126 identified in FIG. 1 is a combination of hardware and software configured to perform specific functionality as described in the following paragraphs.

The API 120 of the server 102 facilitates communication between the client device 104 and the server 102. In some implementations, the API 120 also facilitates communication between the server 102 and the one or more repositories 106. The API 120 packages data packets to (and from) the client device 104, so that there is a bidirectional information flow between the server 102 and the client device 104. The API 120 can package information (e.g., the data records or reference material) received from the client device 104 so that these provided information can be processed by the server 102. The API 120 can package the clinical conditions determined by the server 102 such that the clinical conditions are readable by the client device 104. In some implementations, the API 120 is a web service compatible with hypertext transfer protocol (HTTP) and machine-readable file formats such as extensible markup language (XML) and JavaScript object notation (JSON).

The similarity engine 122 of the server 102 is configured to perform a similarity analysis between the patient records 112 and the reference documents 114. As discussed above, the patient records 112 stored in the repository 106 can be received from the client device 104. The similarity engine 122 uses the language model 110 to determine associations and patterns in the patient records 112 that match those in the reference documents 114. In some implementations, the similarity engine 122 uses word embeddings which allow for a more nuanced and contextual understanding of medical terminology. By analyzing the patient records 112 using word embeddings, the similarity engine 122 using the language model 110 can detect subtle patterns and associations in the patient data that may not be apparent to human experts. Detecting these subtle patterns can lead to faster and more accurate diagnoses of diseases.

In some implementations, the language model 110 is trained on health vocabulary. The health vocabulary is included in the reference documents 114. The similarity engine 122 can use the language model 110 and cosine similarity to compare the reference documents 114 and the patient records 112 to identify the clinical conditions. The reference documents 114 may include one or more clinical conditions and information associated with the one or more clinical conditions. The information can include symptoms associated with each of the one or more clinical conditions. The similarity engine 122 can use the language model 110 to match symptoms of the patient records 112 to the reference documents 114 in order to identify clinical conditions present in the reference documents 114 that match the symptoms.

In an example, the language model 110 can include weights for an artificial neural network, parameters for a regression or classification model, etc. The similarity engine 122 uses the language model 110 to determine the clinical conditions. The clinical conditions can be ranked based on a score (e.g., a confidence score, a cosine similarity score, etc.) for each of the identified clinical conditions.

The scoring engine 124 of the server 102 can determine feature scores that contributed to the cosine similarity scores. In some implementations the feature score is a Shapley value. In some implementations, the feature score is a SHapley Additive explanations (SHAP) value. A SHAP value for a specific feature shows how much the specific feature contributes, either positive or negatively, to a specific clinical condition having a specific cosine similarity score. Examples of features include symptoms exhibited by the patient, an age of the patient, locations that the patient has visited, parasites associated with the locations, diseases common within the locations, etc.

The SHAP value is used on words or phrases in the patient records 112 to determine which of those words or phrases were most important in contributing to the cosine similarity scores for each of the identified conditions in the reference documents 114. For example, words or phrases like “obesity,” “insulin resistance,” and “polyuria” (excessive urination) can be found to be particularly important in contributing to the cosine similarity scores for Type 2 diabetes. In contrast, words or phrases like “autoimmune,” “pancreatic beta cells,” and “ketosis” can be found to be important in contributing to the similarity scores for Type 1 diabetes. Thus, if the similarity engine 122 provides clinical conditions of Type 1 and Type 2 diabetes, and cosine similarity scores associated with both, then the SHAP values can be used to determine which words in the patient records 112 contributed to each of the cosine similarity scores of Type 1 and Type 2 diabetes diagnoses. The provision of the SHAP values by the scoring engine 124 can help medical professionals understand the decision-making process of the similarity engine 122 with the current language model 110. This insight can help provide more accurate and informed diagnoses.

In some implementations, the SHAP values can be arranged by magnitude, thus allowing physicians and doctors to determine top contributors or symptoms that influenced the chosen clinical conditions. In some implementations, features with the top three scores, features with the top four scores, features with the top five scores, etc., are considered by the physician. In some implementations, features can be demoted or promoted based on feedback provided by the physician.

The reinforcement learning engine 126 of the server 102 manages algorithms for determining signals used for re-training or adjusting the language model 110 based on the feedback from the physician (e.g., the user of the client device 104). In some implementations, the algorithm used for reinforcement learning is the policy gradient method where policy parameters are adjusted in the direction of increasing rewards. For example, for a given interaction with the system 100, the server 102 can provide a ranking of six features with the top six scores that led to selecting a specific condition. The user of the client device 104 can indicate that the ranking is incorrect and a third-ranked feature should be ranked higher (i.e., promoted). The reinforcement learning engine 126 can record the reward (e.g., the reward can be derived from the human feedback and can be a simple +1 if the ranking is correct and a −1 if the ranking is incorrect).

The reinforcement learning engine 126 uses the policy gradient method to maximize an expected reward with respect to policy parameters θ. Eqn. 1 provides a gradient of the expected reward with respect to the policy parameters θ. In Eqn. 1, a is the learning rate, πθ(α|s) is the policy which defines the probability of taking action a in state s under policy parameters θ, and Gₜ is the cumulative reward from timestep t.

$$Δθ = α ∇ θ log πθ ( a \vert s ) {G}_{t} \qquad Eqn . 1$$

In the policy gradient method, as applied by the reinforcement learning engine 126, actions correspond to the ranking of features. The policy πθ(α|s) represents the probability of assigning a specific ranking to a feature, given the current state of the language model 110 and the policy parameters θ. The policy parameters can correspond to the weights in the word embeddings. With each iteration, the ranking of the features (i.e., the actions) that led to higher rewards would become more probable, leading to an improvement in the accuracy of the language model over time. In an example, if a medical professional provides feedback indicating that a certain term or phrase should have a higher contribution towards the diagnosis of a specific disease, this feedback can be treated as a reward to promote the ranking of the term or phrase in future predictions. Conversely, if feedback indicates that a certain term or phrase was not relevant for a given diagnosis, the term or phrase can be demoted.

Thus, the reinforcement learning engine 126 can thus use the results from the similarity engine 122 and the scoring engine 124 to determine how to adjust the learning model 110. For example, the similarity engine 124 can provide a list of diagnoses, and the scoring engine 124 can rank, for each diagnosis, words or phrases that contributed to the diagnosis. The rankings provided constitute the action for the reinforcement learning engine 126. The language model 110 used to determine the current rankings indicate the state, and the weights of word embeddings constitutes the policy parameters. The reinforcement learning engine 126 can use the action, the state, the policy parameters, and feedback provided by the user of the client device 104 in Eqn. 1 to determine how to adjust the weights of the word embeddings in the language model 110. The server 102 can thus dynamically and iteratively adjust the language model 110 based on human feedback, continually improving its accuracy and effectiveness in making predictions.

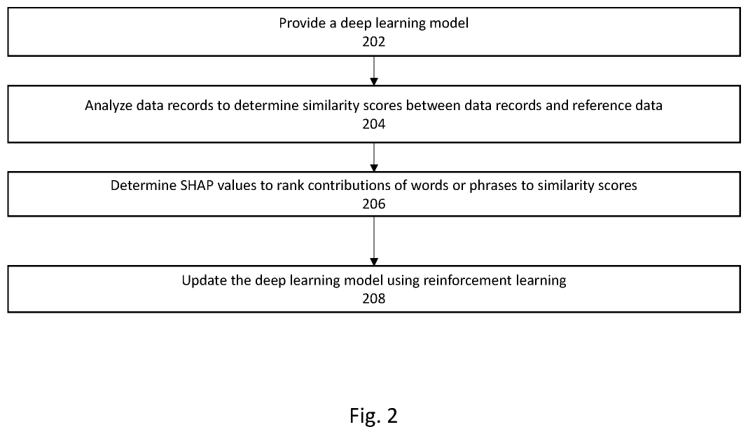

FIG. 2 illustrates a process 200 for training a machine learning model (e.g., the language model 112 of FIG. 1), according to some implementations of the present disclosure. The process 200 is performed by the server 102. At step 202, the server 102 provides a deep learning model (e.g., the language model 112). The language model 112 can be a large language model (e.g., GPT-4). The language model 112 can be received from the repository 106 and/or the client device 102. The server 102 also receives data records associated with a patient (e.g., the patient records 114 or portions of the patient records 114). The server 102 further receives reference data. The reference data are reference documents 116. In some implementations, the reference data includes health-related vocabulary.

At step 204, the server 102 analyzes the data records to determine similarity scores between the data records and the reference data. In some implementations, the similarity scores are cosine similarity scores. In some implementations, the reference data includes multiple excerpts arranged by chapters or sections such that each section pertains to a separate disease or condition. Diseases can be coded or arranged by code, with each section corresponding to a different code (e.g., a different ICD-10 code). For example, a first section can be directed at Type 1 diabetes, and a second section can be directed at Type 2 diabetes. The first section can include one or more codes identifying the first section as being directed at Type 1 diabetes. The second section can include one or more codes identifying the second section as being directed at Type 2 diabetes. The server 102 analyzes the data records to determine a similarity between the contents of the first section and the data records to provide a first similarity score. The server 102 analyzes the data records to determine a similarity between the contents of the second section and the data records to provide a second similarity score. That is, using the language model 112 (e.g., a large language model) and the reference data, the server 102 can determine similarity scores for each section.

In some implementations, when large reference data including hundreds or thousands of sections are used for comparison, only similarity scores exceeding a similarity score threshold are returned. For example, comparing a patient's longitudinal health record against reference data consisting of sections on Type 1 diabetes, Type 2 diabetes, and arthritis can provide similarity scores of 0.9, 0.8, and 0.3, respectively. If the similarity score threshold is set at 0.7, then only sections on Type 1 diabetes and Type 2 diabetes will be returned. The use of similarity score thresholds can help limit the number of predicted diagnoses in some situations.

In determining the similarity scores, the server 102 uses word embeddings determined using the language model 110. The word embeddings allow comparing the words and phrases in the data records to the words and phrases in the reference data. Word embeddings allow comparing words that may not look the same but have similar meanings. This is advantageous because subtle differences and meanings between words can be captured. Meanings of compound words and phrases can also be captured. Synonyms can be captured as well, thus allowing a better diagnosis when compared with a purely text-based searching. Furthermore, comparing word embeddings in vector space allows using cosine similarities to provide quantitative comparisons rather than qualitative comparisons. In the example above, the quantitative comparisons allow the server 102 to determine which of the first section or the second section is a closer match to the data record based on cosine similarity values returned for the first section and the second section. The higher the cosine similarity values, the better the match. In some implementations, the cosine similarity value is quoted as a value between 0 and 1. The server 102 can rank the conditions based on the cosine similarity values. For example, Type 1 diabetes can be provided first with its corresponding cosine similarity value before Type 2 diabetes is provided with its lower corresponding cosine similarity value.

In some implementations, the data records are longitudinal health records. Longitudinal health records can include informal notes from healthcare professionals that can contain information that may signify some illness. The illnesses may be transitory or chronic and past or present. The longitudinal health records can contain information for identifying comorbidities. Typically, the longitudinal health records contain data that should be further analyzed to identify illnesses and may not outright list an illness. For example, the longitudinal health records can include blood pressure information at specific dates, symptoms suffered at specific dates, observation or notes by a physician or caretaker indicating treatments and/or patient habits or behavior, patient allergies, etc. This information can holistically be associated with one or more illnesses. Embodiments of the present disclosure use deep learning based semantic text matching with word vectors and embedding to identify illnesses or other conditions from a patient's health-related data. Health-related data includes the longitudinal health records, demographic information, drug information, or any other information that can be included in a patient's electronic health record. Health-related data can also include results from clinical trails, journal papers, clinical notes, etc. The health-related data can be pre-processed for deep learning analysis. Pre-processing the longitudinal patient record includes removing any personally identifying information and converting the record into a format that is inputted into the large language model.

In an example, if the language model 110 is a large language model like GPT-4 or BERT, word embeddings are used to represent the patient's longitudinal health record as a vector. The cosine similarity between this vector and pre-computed vectors for each possible diagnosis are used to determine the most likely diagnosis. In some cases, a confidence score is computed to indicate the accuracy of the diagnosis. In a diabetes example, E10 is Type 1 diabetes mellitus, E11 is Type 2 diabetes mellitus, E13 is other specified diabetes mellitus, E08 is diabetes mellitus due to underlying condition, and E09 is drug or chemical induced diabetes mellitus. To separate ICD10 code E11 from E10, E13, E08, and E09 in word embedding space, the language model 110 was previously trained on medical texts to learn word embeddings that capture the semantic relationships between different medical terms. The cosine similarity between the vector and pre-computed vectors for each possible type of diabetes can be used to determine the most likely diabetes diagnosis.

The language model 110 is fine-tuned on a dataset of longitudinal patient records labeled with the correct diagnosis, including both E11 and other types of diabetes, as well as underlying conditions and drug-induced diabetes. The language model 110 is used to compute word embeddings for the preprocessed patient record. The cosine similarity between the patient record embeddings and pre-computed embeddings is computed for each possible diagnosis (E11, E10, E13, E08, and E09). In some implementations, confidence scores are computed based on the cosine similarities. Any diagnoses with a low confidence score can be eliminated, based on established diagnostic criteria for each condition. In some implementations, the server 102 provides the most likely diagnosis based on the highest cosine similarity score, along with the confidence score indicating the accuracy of the diagnosis.

At step 206, the server 102 determines feature scores for ranking contributions of words or phrases to the cosine similarity scores. The scoring engine 124 can determine the feature scores by using the SHAP method. For example, given that step 204 provides the cosine similarity scores between the data records and the reference data, the scoring engine 124 can provide sets of words that contributed to the specific cosine similarity scores. For example, given the following cosine similarity scores from step 204: {condition 1, cosine similarity score 1; condition 2, cosine similarity score 2; condition 3, cosine similarity score 3; . . . condition n, cosine similarity score n}. The scoring engine 124 can provide {word₁ 1, word₁ 2, . . . word₁ p; word₂ 1, word₂ 2, . . . word₂ q; word₃ 1, word₃ 2, . . . word₃ r; . . . word_{condition n} 1, word_{condition n} 2, . . . word_{condition n} s}. p is an integer corresponding to a total number of words associated with condition 1, q is an integer corresponding to a total number of words associated with condition 2, r is an integer corresponding to a total number of words associated with condition 3, and s is an integer corresponding to a total number of words associated with condition n. In some implementations, q, r, and s are the same number. For example, for each of the conditions, the words with the highest SHAP values are provided. The three, four, five, six, etc., words with the highest SHAP values can be provided. In some implementations, q, r and s are different numbers. For example, for each of the conditions, the words with magnitudes of SHAP values that exceed a certain SHAP value threshold are provided. The words with SHAP value magnitudes that exceed SHAP value thresholds of, for example, 0.4, 0.5, 0.6, 0.7, etc., are provided. The SHAP values can range from −1 to 1.

At step 208, the server 102, in conjunction with the client device 104, updates the language model 110 using reinforcement learning. The language model 110 includes vocabulary and synonyms that are not explicitly disclosed in the data records. The language model 110 can be updated over time based on reinforcement learning in order to improve accuracy of the word embeddings of step 204. The language model 110 is iteratively and dynamically adjusted or updated using a human-in-the-loop based reinforcement learning from human feedback (RLHF). Human-in-the-loop RLHF can involve receiving from a user of the client device 104, via an electronic human-machine interface (HMI), an input representative of a modification to at least one of the contributions determined by the SHAP computation.

Human-in-the-loop RLHF further involves responsive to receiving the input, adjusting at least one of the ranks of the word embeddings in the model. RLHF is a unique feature because RLHF allows the server 102 to continuously learn and improve the accuracy of the language model 110 over time, making the language model 110 more effective and efficient. For example, a medical professional may review the potential diagnoses and differential diagnoses generated by using the language model 110 and provide feedback on which diagnoses are more or less likely, based on the medical professional's experience and knowledge. The use of SHAP-based explainability is also unique, providing a transparent and trustworthy way to understand how the server 102 uses the language model 110 in making predictions. The transparency helps medical professionals build trust and confidence in the learning model 110. The server 102 can improve patient outcomes in ways conventional methods cannot.

In some implementations, human-in-the-loop RLHF involves using a policy gradient method for reinforcement learning. For example, at step 206, the server 102 provides, to the client device 104, SHAP values and ranks words or phrases for a first condition. The user of the client device 104, for example, a healthcare professional, examines the ranking of words or phrases that contributed to the prediction of the first condition. That is, the user of the client device 104 examines the prediction of system 100. The initial state s for use in Eqn. 1 can be represented by the patient records 112 processed into word embeddings and the language model 110. Note that the language model 110 previously incorporates word embeddings of the reference documents 114. The patient records 112 have been transformed into a sequence of word embeddings, with weights θ (i.e., policy parameters). θ is a high-dimensional vector that parameterizes the language model 110. Each word or phrase in the clinical notes can be seen as an action a taken by the policy (i.e., an action a taken by the language model 110).

The policy πθ(α|s) dictates the probability of a word or phrase being ranked in a certain way given the current state s and weights θ in the language model 110. Initially, the policy πθ(α|s) is determined by the existing configuration of the language model 110. The rankings initially provided at step 206 are reviewed by the healthcare professional, and the healthcare professional provides feedback to the server 102.

The feedback provided to the server 102 is equivalent to a reward signal r. The reward signal r is positive if the prediction is correct and negative if the prediction is incorrect. The total reward or return Gₜ can be calculated. In some implementations, Gₜ is determined to be the immediate reward r, since in the current context, each prediction can be considered as a separate episode. In some implementations, Gₜ can exhibit a more complex computation where multiple episodes are considered as a sequence of predictions. In these cases, Gₜ can be formulated as a sum of future rewards, potentially discounted by a factor γ.

The feedback is used to update weights θ (or policy parameters) of the language model 110. Policy gradient method of Eqn. 1 can be used to determine weight updates Δθ. The gradient ∇θ log πθ(α|s) provides the direction of the steepest ascent in the parameter space that would increase the log likelihood of the action taken. In Eqn. 1, this direction is scaled by the total reward Gₜ, meaning hat if the return is positive (good feedback indication), then the likelihood of such action in a similar state will increase for future predictions. If the return is negative (bad feedback indication), the likelihood of such action will decrease. After several iterations of this process, the language model 110 learns to assign higher ranks to words or phrases that consistently receive positive feedback, improving the accuracy of its predictions over time.

Thus, using reinforcement learning, initially, the server 102 can use the reinforcement learning engine 126 to fine-tune the language model 110 to a specifically tailored dataset (i.e., the reference documents 114). In an example, the language model 110 is a GPT-4 model that is fine-tuned on the DSM-5.

In some implementations, the reinforcement learning engine 126 can use actor-critic framework in adjusting the language model 110 to better match medical professionals' knowledge and experience. For example, in a first step, the language model 110 is initially trained by adjusting parameters θ of the language model 110 to better suit the context of clinical conditions from health records.

In a second step, a reward model can be created. Different outputs of the language model 110 can be observed for the same input. For example, the patient records 112 can be provided to the initially trained language model 110 to observe the different outputs of the initially trained language model 110. Potential diagnoses (i.e., outputs) of the language model 110 can be ranked and assigned scores based on their relevancy and accuracy. This scoring trains a reward model to evaluate the language model 110 outputs in a manner consistent with expert human judgment.

In a third step, Proximal Policy Optimization (PPO) reinforcement learning algorithm can be performed by the server 102 using a two-step process—a “make experience” stage and a “parameter update” stage. In the “make experience” stage, the supervised fine-tuning, actor, reward, and Critic models calculate generated experiences. The actor, reward, and critic models can be separate models implemented by the reinforcement learning engine 126. These generated experiences include states, actions, and rewards, which are stored in an experience buffer. The experience buffer can be provided in the server 102. In the “parameter update” stage, policy and value losses are calculated from the experiences in the buffer. Policy loss corresponds to the deviation of the predicted diagnoses from the expected ones. Value loss measures the variance in predicted patient outcomes.

In tandem with PPO, the server 102 performs a process of pretraining gradient mixing (PTX) which calculates a cross-entropy loss between outputs provided by the language model 110 and the actual diagnosis in the input corpus (i.e., the reference documents 114). The overall loss or total loss Lₜₒₜₐₗ of Eqn. 2 is then calculated as the sum of policy loss L_{policy}, value loss Lᵥₐₗᵤₑ, and PTX loss L_{PTX}. This total loss Lₜₒₜₐₗ is used by the server 102 to update the parameters of the language model 110 using backpropagation. By minimizing the total loss Lₜₒₜₐₗ, the language model 110 can dynamically learn and enhance its accuracy over time, thereby ensuring more effective and efficient identification of clinical conditions from health records.

$${L}_{total} = {L}_{policy} + {L}_{value} + {L}_{PTX} \qquad Eqn . 2$$

By incorporating human expert feedback into the RLHF process, embodiments of the present disclosure can adjust interpretations of language learning models (e.g., the language model 110) to align more closely with medical professionals' knowledge and experience, ultimately leading to improved patient outcomes. RLHF is used creatively to optimize a specific language model for interpreting and understanding medical data.

Embodiments of the present disclosure can be used to improve outputs of a language learning model for a specific domain. The outputs of the language learning model can be ranked. For example, the language learning model can receive as input a patient's longitudinal health record and provide as output a ranking of diagnoses. Feedback can be provided to promote or demote a specific diagnosis within the ranking, and the feedback provided can be used to update the language learning model to improve the accuracy of the language learning model. Alternatively, SHAP values can be determined to extract, in order, features that contributed to the ranking. The features can be promoted or demoted via feedback. The feedback can be used to update the language learning model to improve the accuracy of the language learning model.

The following paragraphs illustrate some capabilities of some embodiments of the present disclosure.

### Example 1

Doctor's Note: Patient presented with complaints of increased thirst and frequent urination, as well as fatigue and blurry vision. Physical examination revealed a body mass index (BMI) of 31 and elevated blood glucose levels. Lab results showed fasting blood glucose of 170 mg/dL and HbA1c of 8.5%, as well as hypertriglyceridemia and low HDL cholesterol. Microalbuminuria was also present, indicating early kidney damage. Subsequent testing showed early diabetic retinopathy, peripheral neuropathy, and early osteoporosis. Despite treatment with metformin, insulin, and an SGLT2 inhibitor, blood glucose levels and HbA1c remained elevated. Urine microalbumin and kidney function tests indicated progression of kidney damage, while lipid panel results showed hypercholesterolemia and low HDL cholesterol. Foot exam showed no signs of neuropathy or ulcers, and colonoscopy showed no abnormalities.

The Doctor's Note above does not mention “diabetes mellitus,” but based on the patient's symptoms and lab results, there is a strong suspicion that a metabolic disorder, likely Type 2 diabetes (E11), is present. Further monitoring and management will be necessary to prevent further complications associated with the condition.

At step 204, word embedding and distance based similarity analysis is performed by comparing the meaning of words based on their relative positions in a high-dimensional vector space. In the Doctor's Note, word embeddings are used to identify terms that are associated with diabetes mellitus, such as, “increased thirst,” “frequent urination,” “elevated blood glucose levels,” “HbA1c,” “microalbuminuria,” “diabetic retinopathy,” “peripheral neuropathy,” and “elevated lipid panel results.”

Based on the cosine similarity between the word embeddings of these terms and the patient's medical record, the server 102 determines the degree of similarity between the patient's symptoms and those associated with diabetes mellitus. The higher the cosine similarity score, the more likely it is that the patient has diabetes mellitus. In this specific case, the patient's medical record contains several key terms that are highly associated with diabetes mellitus, including “increased thirst,” “frequent urination,” “elevated blood glucose levels,” “HbA1c,” “microalbuminuria,” “diabetic retinopathy,” and “peripheral neuropathy.” This indicates a strong similarity between the patient's symptoms and those associated with diabetes mellitus. Therefore, based on the word embedding distance-based similarity analysis, it is highly likely that the patient has diabetes mellitus.

### Example 2

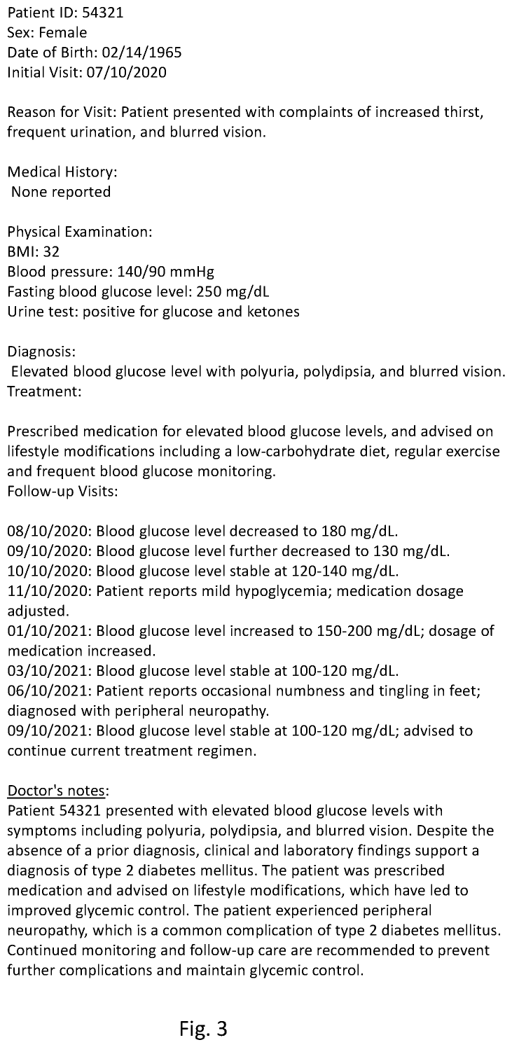

Adjusting the example a bit with a longitudinal patient record, the Doctor's Note is provided for a patient that has the following longitudinal patient record provided in FIG. 3. The process 200 will find that Type 2 diabetes (E11) is the most likely diagnosis. This is due to the following information:
1. E10—Type 1 diabetes mellitus: This code is used for patients with Type 1 diabetes, which is a chronic condition in which the pancreas produces little or no insulin.
2. E11—Type 2 diabetes mellitus: This code is used for patients with Type 2 diabetes, which is a chronic condition in which the body either resists the effects of insulin or doesn't produce enough insulin to maintain normal glucose levels.
3. E13—Other specified diabetes mellitus: This code is used for patients with other types of diabetes, such as gestational diabetes, drug-induced diabetes, or other types of diabetes not classified elsewhere.
4. E08—Diabetes mellitus due to underlying condition: This code is used for patients with diabetes that is caused by an underlying condition, such as pancreatic disease or endocrine disorders.
5. E09—Drug or chemical induced diabetes mellitus: This code is used for patients with diabetes that is caused by drugs or chemicals, such as corticosteroids or diuretics.
6. In addition to the above codes, there are also codes that indicate specific complications or manifestations of diabetes, such as diabetic retinopathy (E11.3), diabetic neuropathy (E11.4), diabetic foot ulcer (E11.6), and diabetic ketoacidosis (E10.1 or E11.1). These codes are used to provide additional information about the patient's condition and to ensure appropriate billing and reimbursement for healthcare services.

By applying the process 200 to the patient's longitudinal record, the server 102 will likely assign a high probability score to the diagnosis of Type 2 diabetes mellitus based on the patient's symptoms and laboratory findings.
1. The longitudinal patient record for a patient with undiagnosed E11 (type 2 diabetes mellitus) would likely eliminate other types of diabetes (E10, E13) as well as drug-induced or underlying conditions (E08, E09) based on several factors:
2. Symptoms: Type 1 diabetes (E10) typically presents with more severe symptoms, such as weight loss, nausea, and vomiting, and may develop more quickly than type 2 diabetes. The patient in the longitudinal record presented with symptoms of increased thirst, frequent urination, and blurred vision, which are more commonly associated with type 2 diabetes.
3. Age: Type 1 diabetes is typically diagnosed in childhood or adolescence, while type 2 diabetes is more common in adults. The patient in the longitudinal record was born in 1965, making type 2 diabetes more likely.
4. Family history: Type 2 diabetes has a stronger genetic component than type 1 diabetes. The patient in the longitudinal record had no reported medical history or family history of diabetes, making type 1 diabetes less likely.
5. Response to treatment: Type 2 diabetes often be managed with medication and lifestyle modifications, while type 1 diabetes typically requires insulin therapy. The patient in the longitudinal record responded well to medication and lifestyle modifications, suggesting type 2 diabetes.

Based on these factors, an algorithm using machine learning or natural language processing techniques could analyze the patient's longitudinal record and assign a high probability score to the diagnosis of type 2 diabetes (E11), while eliminating other potential diagnoses such as type 1 diabetes (E10), other specified diabetes (E13), diabetes due to underlying conditions (E08), or drug-induced diabetes (E09). The algorithm could also generate a report explaining the rationale behind the diagnosis and the factors used to eliminate other potential diagnoses.

In some implementations, in step 206, SHAP values can be used to explain why other types of diabetes were eliminated. SHAP values are used to explain the contribution of each feature to the final output (i.e., cosine similarity with the highest outcome) of the server 102. For example, in the case of the longitudinal patient record for a patient with undiagnosed E11, SHAP values can be used to explain why other types of diabetes (E10, E13) as well as drug-induced or underlying conditions (E08, E09) were eliminated as potential diagnoses.

SHAP values can assign a feature score to each feature in the longitudinal record, indicating how much each feature contributed to the final prediction of type 2 diabetes. The features that contributed the most to the prediction will have higher SHAP values, while features that had little or no impact on the prediction will have lower SHAP values. For example, the SHAP values for the patient's age would likely be relatively low, as age alone does not provide enough information to distinguish between different types of diabetes. The patient's symptoms of increased thirst, frequent urination, and blurred vision would likely have higher SHAP values, as these are more strongly associated with type 2 diabetes. Family history, response to treatment, and other factors could also be assigned SHAP values to help explain the final prediction.

By using SHAP values to explain the output of the machine learning model, healthcare providers better understand the factors that contribute to a patient's diagnosis and treatment plan. This helps improve the accuracy of diagnoses and provide more personalized care to patients.

### Example 3

In a case of the longitudinal patient record for a patient with undiagnosed E11, the large language model can likely assign a high cosine similarity score to the diagnosis of type 2 diabetes mellitus (E11) based on the patient's symptoms and medical history. The model could then eliminate other potential diagnoses based on the absence of specific symptoms or diagnostic criteria associated with those conditions.

In some implementations, the learning model 110 is trained with a word embedding model using medical texts such as research papers, clinical notes, and textbooks. The language model 110 can capture the semantic meaning of medical terms, such as symptoms, diagnoses, treatments, and medications, as word embeddings. In the diabetes example, because the embeddings are learned using a large medical text corpus, the cosine similarity between E11 and E10 should be higher than the similarity between E11 and other codes, indicating that E11 is more closely related to Type 2 diabetes mellitus (E11) than to other types of diabetes or underlying conditions.

Distance in word embeddings helps with differential diagnosis by measuring the similarity between the patient's symptoms and different medical conditions. By representing medical terms as vectors in a high-dimensional space, the server 102 can compute the distance between these vectors to quantify the similarity between different medical conditions. This allows the comparison of the patient's symptoms with the symptoms associated with different medical conditions and identify the condition that is most likely to be causing the patient's symptoms. The closer the patient's symptoms are to the symptoms associated with a specific medical condition in the word embedding space, the more likely the patient has that condition.

The word embedding model can identify the following words as being semantically similar to E11 than to E10, E13, E08, and E09: Obesity, Insulin resistance, Hyperglycemia, Metformin, Glucose intolerance, Diabetic retinopathy, Polyuria, Polydipsia, Neuropathy, Atherosclerosis. To find terms that are semantically similar to E10, E13, E08, and E09 but dissimilar to E11, a word embedding model is used to compute the cosine similarity between the embeddings for each code and other medical terms. The terms with the highest cosine similarity to each code and the lowest cosine similarity to E11 would be the ones that are most semantically similar to E10, E13, E08, and E09.

Here are some terms that may be more semantically similar to E10, E13, E08, and E09 than to E11 based on a word embedding model:

E10 (Type 1 diabetes mellitus)
1. Autoimmune
2. C-peptide
3. Islet cells
4. Diabetic ketoacidosis
5. Insulin pump

E13 (Other specified diabetes mellitus)
1. Hyperosmolarity
2. Secondary diabetes
3. Steroid-induced diabetes
4. Pancreatitis
5. Monogenic diabetes

E08 (Diabetes mellitus due to underlying condition)
1. Cushing's syndrome
2. Acromegaly
3. Hyperthyroidism
4. Hemochromatosis
5. Chronic pancreatitis

E09 (Drug or chemical induced diabetes mellitus)
1. Steroids
2. Antipsychotics
3. Diuretics
4. Beta blockers
5. HIV medication

According to some embodiments of the present disclosure, processes described above with reference to flow charts or flow diagrams (e.g., in FIG. 2) may be implemented in a computer software program. For example, some embodiments of the present disclosure include a computer program product, which includes a computer program that is carried in a computer readable medium. The computer program includes program codes for executing the method 200. The computer program may be downloaded and installed from a network (e.g., the Internet, a local network, etc.) and/or may be installed from a removable medium (e.g., a removable hard drive, a flash drive, an external drive, etc.). The computer program, when executed by a central processing unit implements the above functions defined by methods and flow diagrams provided herein in the present disclosure.

A computer readable medium according to the present disclosure may be a computer readable signal medium or a computer readable storage medium or any combination of the above two. Examples of the computer readable storage medium may include electric, magnetic, optical, electromagnetic, infrared, or semiconductor systems, elements, apparatuses, or a combination of any of the above. More specific examples of the computer readable storage medium include a portable computer disk, a hard disk, a random access memory (RAM), a read only memory (ROM), an erasable programmable read only memory (EPROM or flash memory), an optical fiber, a portable compact disk read only memory (CD-ROM), an optical memory, a magnetic memory, or any suitable combination of the above.

The computer readable storage medium according to some embodiments may be any tangible medium containing or storing programs, which may be used by, or used in combination with, a command execution system, apparatus or element. In some embodiments of the present disclosure, the computer readable signal medium may include a data signal in the base band or propagating as a part of a carrier wave, in which computer readable program codes are carried. The propagating data signal may take various forms, including but not limited to an electromagnetic signal, an optical signal, or any suitable combination of the above. The computer readable signal medium may also be any computer readable medium except for the computer readable storage medium. The computer readable medium is capable of transmitting, propagating or transferring programs for use by, or used in combination with, a command execution system, apparatus or element. The program codes contained on the computer readable medium may be transmitted with any suitable medium, including but not limited to: wireless, wired, optical cable, RF medium, etc., or any suitable combination of the above.

A computer program code for executing operations in the present disclosure may be compiled using one or more programming languages or combinations thereof. The programming languages include object-oriented programming languages, such as Java or C++, and also include conventional procedural programming languages, such as “C” language or similar programming languages. The program code may be completely executed on a user's computer, partially executed on a user's computer, executed as a separate software package, partially executed on a user's computer and partially executed on a remote computer, or completely executed on a remote computer or electronic device. In the circumstance involving a remote computer, the remote computer may be connected to a user's computer through any network, including local area network (LAN) or wide area network (WAN), or be connected to an external computer (for example, connected through the Internet using an Internet service provider).

The flow charts and block diagrams in the accompanying drawings illustrate architectures, functions and operations that may be implemented according to the systems, methods and computer program products of the various embodiments of the present disclosure. Each of the blocks in the flow charts or block diagrams may represent a program segment or code that includes one or more executable instructions for implementing specified logical functions. It should be further noted that, in some alternative implementations, the functions denoted by the flow charts and block diagrams may also occur in a sequence different from the sequences shown in the figures. For example, any two blocks presented in succession may be executed substantially in parallel, or sometimes be executed in a reverse sequence, depending on the functions involved. It should be further noted that each block in the block diagrams and/or flow charts as well as a combination of blocks in the block diagrams and/or flow charts may be implemented using a dedicated hardware-based system executing specified functions or operations, or by a combination of dedicated hardware and computer instructions.

Engines, handlers, generators, managers, or any other software block or hybrid hardware-software block identified in some embodiments of the present disclosure may be implemented by software, or may be implemented by hardware. The described blocks may also be provided in a processor.

While the present disclosure has been described with reference to one or more particular implementations, those skilled in the art will recognize that many changes may be made thereto without departing from the spirit and scope of the present disclosure. Each of these embodiments and implementations and obvious variations thereof is contemplated as falling within the spirit and scope of the present disclosure, which is set forth in the claims that follow.

What is claimed is:

1. A system for conducting feature importance analysis, the system comprising a processor and a non-transitory computer readable medium storing instructions such that when the instructions are executed by the processor, the system is configured to:
provide a deep learning model;
analyze data records using word embeddings to produce vectors indicative of similarity scores for words or phrases in the data records relative to electronically stored reference data;
compute shapeley additive explanation (SHAP) values to analyze and rank contributions of the words or phrases to the similarity scores from the word embeddings;
use a language model trained on a vocabulary used in the data records to identify contextual insights that are not explicitly disclosed in the data to improve accuracy of the word embeddings; and
iteratively and dynamically adjust or update the model using human-in-the-loop based reinforcement learning from human feedback (RLHF) by:
receiving from a human via an electronic human-machine interface (HMI) an input representative of a modification to at least one of the contributions determined by the SHAP computation; and
responsive to receiving the input, adjusting at least one of the ranks of the word embeddings in the model.

2. The system of claim 1, wherein the data records include longitudinal patient data, and wherein the vocabulary used in the data records is a healthcare vocabulary, the system further configured to: pre-process the data records to prepare the data records for deep learning analysis by the deep learning model.

3. The system of claim 2, further configured to: responsive to analyzing the data records, automatically identify a disease from clinical notes in the data records by comparing the clinical notes to the healthcare vocabulary using sematic text matching with the vectors and the word embeddings.

4. The system of claim 3, wherein the disease includes Type 1 diabetes mellitus, Type 2 diabetes mellitus, or any combination thereof.

5. The system of claim 1, further configured to: determine a confidence score associated with the similarity score.

6. The system of claim 5, wherein the confidence score is based at least in part on the similarity score.

7. The system of claim 5, wherein the confidence score is used to eliminate some of the vectors indicative of the similarity score.

8. The system of claim 5, wherein the confidence score indicates an accuracy of a diagnosis.

9. The system of claim 1, further configured to: determine the rank contributions of the words or phrases based on a threshold associated with the SHAP values.

10. The system of claim 1, further configured to: determine the rank contributions of the words or phrases based on a number of the words or phrases.

11. The system of claim 1, wherein the responsive to receiving the input, adjusting at least one of the ranks of the word embeddings in the model includes determining a total reward for adjusting weights associated with one or more of the word embeddings as represented in the language model.

12. The system of claim 11, wherein the total reward is an immediate reward provided as the input representative of the modification.

13. The system of claim 11, wherein the total reward is determined via a reward network.

14. The system of claim 1, wherein the input representative of the modification is one of a correct feedback indication or an incorrect feedback indication.

15. The system of claim 1, wherein the responsive to receiving the input, adjusting at least one of the ranks of the word embeddings in the model includes updating parameters of the language model via backpropagation.

16. A method for conducting feature importance analysis, comprising:
providing a deep learning model;
analyzing data records using word embeddings to produce vectors indicative of similarity scores for words or phrases in the data records relative to electronically stored reference data;
computing shapeley additive explanation (SHAP) values to analyze and rank contributions of the words or phrases to the similarity scores from the word embeddings;
using a language model trained on a vocabulary used in the data records to identify contextual insights that are not explicitly disclosed in the data to improve accuracy of the word embeddings; and
iteratively and dynamically adjusting or updating the model using human-in-the-loop based reinforcement learning from human feedback (RLHF) by:
receiving from a human via an electronic human-machine interface (HMI) an input representative of a modification to at least one of the contributions determined by the SHAP computation; and
responsive to receiving the input, adjusting at least one of the ranks of the word embeddings in the model.

17. The method of claim 16, wherein the data records include longitudinal patient data, and wherein the vocabulary used in the data records is a healthcare vocabulary, the method further including: pre-processing the data records to prepare the data records for deep learning analysis by the deep learning model.

18. The method of claim 17, further comprising:
responsive to analyzing the data records, automatically identify a disease from clinical notes in the data records by comparing the clinical notes to the healthcare vocabulary using sematic text matching with the vectors and the word embeddings.

19. The method of claim 16, wherein the responsive to receiving the input, adjusting at least one of the ranks of the word embeddings in the model includes determining a total reward for adjusting weights associated with one or more of the word embeddings as represented in the language model.

20. The method of claim 19, wherein the total reward is an immediate reward provided as the input representative of the modification.
